(19) United States
(12) Patent Application Publication
(10) Pub. No.: US 2025/0339580 A1
(43) Pub. Date: Nov. 6, 2025

(54) MEDICAL TISSUE ADHESIVE COMPOSITION

(71) Applicant: OKAPI MEDICAL, LLC, Akron, OH (US)

(72) Inventors: Darren OBRIGKEIT, Aachen (DE); Paul WYMAN, Maastricht (NL)

(21) Appl. No.: 19/187,373

(22) Filed: Apr. 23, 2025

Related U.S. Application Data
(60) Provisional application No. 63/642,618, filed on May 3, 2024.

(51) Int. Cl. A61L 24/06; A61B 17/00

(52) U.S. Cl. CPC A61L 24/06; A61B 17/00491; A61B 2017/00495; A61B 2017/00526

(57) ABSTRACT

A medical adhesive composition particularly suited for use as a wound or surgical incision closure, sealant, adhesive or the like for living tissue. Advantageously, the adhesive composition is applied as a liquid and quickly polymerizes into a durable film, coating, or layer which exhibits desirable properties including, but not limited to, strength, flexibility, moisture vapor transmission and oxygen permeability. Kits including the adhesive composition, an applicator and optionally an adherable substrate such as a mesh are described along with methods for preparing the kits. The adhesive compositions are able to readily hold skin edges of wounds or surgical incisions closed in order to provide a layer of protection against harmful organisms that can cause infection.

### FIELD OF THE INVENTION

The present invention relates to a medical adhesive composition particularly suited for use as a wound or surgical incision closure, sealant, adhesive or the like for living tissue. Advantageously, the adhesive composition is applied as a liquid and quickly polymerizes into a durable film, coating, or layer which exhibits desirable properties including, but not limited to, strength, flexibility, low tack, low formaldehyde migration, moisture vapor transmission and oxygen permeability. Kits including the adhesive composition, an applicator and optionally an adherable substrate such as a mesh are described along with methods for preparing the kits. The adhesive compositions are able to readily hold skin edges of wounds or surgical incisions closed in order to provide a layer of protection against harmful organisms that can cause infection.

### BACKGROUND OF THE INVENTION

Many different medical adhesive compositions have been developed over the years in order to close wounds or surgical incisions created during a medical procedure.

So-called first-generation adhesives were based on compositions including methyl, ethyl, or n-butyl cyanoacrylate. Such acrylates set quickly, but had disadvantages of being brittle, resulting in the heightened potential of the cured composition to fracture, thereby allowing dehiscence and infiltration of harmful organisms. In addition, some of the compositions including cyanoacrylates polymerized too quickly, thereby resulting in thermal injuries to patients due to the exothermic curing reaction.

Some subsequent generations of topical skin adhesives are based on 2-octyl cyanoacrylate. This lower Tg 2-octyl cyanoacrylate was inherently more flexible than prior generation methyl ethyl or, n-butyl homologs. Flexibility of the 2-octyl cyanoacrylate adhesives were further enhanced with the addition of a plasticizer. However, the plasticizer, unreacted monomer, as well as other additives included in the adhesive composition, were found to migrate out of the cured film or coating, one or more of to the patient's tissue and to the outer surface of the film. This phenomenon is sometimes described as “blooming”. It has also been discovered that foreign substances can migrate into skin adhesives based on 2-octyl cyanoacrylate. Migration can result in a tacky surface typical of (very) low Tg materials that may pick up dirt and debris from the ambient environment.

Formaldehyde creation and migration is also a problem with some prior art medical adhesives.

Thermal injuries were reportedly addressed by including heat dissipating agents in the form of biocompatible solvent. However, the inclusion of heat dissipating agents increased the potential for skin reactions in patients who are sensitive to such agents that can migrate from the cured adhesive composition.

Examples of medical adhesive compositions in patent literature include the following:

U.S. Publication No. 2015/0328357 relates to a wound protecting composition includes an end-functionalized arborescent polymer having at least two branching points to provide at least four ends to the arborescent polymer, the arborescent polymer comprising polyisobutylene; and an end group attached to a majority of the ends of the arborescent polymer, the end group comprising an alkyl cyanoacrylate. The composition may further include a blend of the end-functionalized arborescent polymer with an independent alkyl cyanoacrylate.

U.S. Pat. No. 9,603,868 relates to a substantially homogeneous miscible liquid adhesive composition comprising a relatively high number average molecular weight (Mn=6,000-10,000 g/mol) multi-arm star polymer having polyisobutylene chains terminated with cyanoacrylate groups (High-Ø(PIB-CA)3); 2-octyl cyanoacrylate (Oct-CA); and a low boiling point biocompatible solvent having a boiling point of not more than 37° C. When this adhesive composition contacted with living tissue and reacted with a nucleophile, such as water or an initiator, the biocompatible solvent will evaporate and the High-Ø(PIB-CA)3 and Oct-CA will co-polymerize in-situ to form a poly(2-octyl cyanoacrylate)-polyisobutylene co-network suitable for any of a number of biomedical applications, from wound closure and healing of skin tissue, to sealant for surgical cuts.

U.S. Publication No. 2016/0215098 relates to a family of silicone rubbers (e.g., polydimethylsiloxane) carrying one or more cyanoacrylate groups and their methods of production are provided. These silicone rubbers endowed with cyanoacrylate groups are useful in a variety of wound care applications, including wound closures, adhesives, sealants, and skin protectors. The silicone rubber moiety provides oxygen and moisture permeability (i.e., “breathability”), biocompatibility, optical transparency, and good mechanical properties, while the cyanoacrylate group imparts instantaneous adhesion/attachment to living tissues such as skin on par with that of contemporary cyanoacrylate-based wound care products.

U.S. Publication No. 2017/0136058 relates to a substantially homogeneous miscible liquid adhesive composition comprising a relatively high number average molecular weight (Mn=6,000-10,000 g/mole) cyanoacrylate tri-telechelic star polymer having polyisobutylene chains terminated with cyanoacrylate groups (High-Ø(PIB-CA)3); 2-octyl cyanoacrylate (Oct-CA); and a relatively low molecular weight (Mn=1,000-4,000 g/mole) cyanoacrylate tri-telechelic star polymer having polyisobutylene chains terminated with cyanoacrylate groups (Low-Ø(PIB-CA)3). The Low-Ø(PIB-CA)3 compatibilizes the High-Ø(PIB-CA)3 and Oct-CA removing the need for a solvent. When the substantially homogeneous miscible liquid adhesive compositions of various embodiments are reacted with a nucleophile, such as water or an initiator, they form a polymer co-network suitable for any of a number of biomedical applications, from wound closure and healing of skin tissue, to sealant for surgical cuts.

U.S. Publication No. 2017/0028099 relates to a method for increasing the rate of polymerization of 2-octyl cyanoacrylate, or the rate of copolymerization of 2-octyl cyanoacrylate and a tri-telechelic star polymer comprising polyisobutylene terminated with cyanoacrylate groups (Ø(PIB-CA)₃) to form a co-network, is provided. The method comprises initiating the polymerization of 2-octyl cyanoacrylate, or the copolymerization of 2-octyl cyanoacrylate and a tri-telechelic star polymer comprising polyisobutylene terminated with cyanoacrylate groups (Ø(PIB-CA)₃) to form the co-network, with an initiator selected from the group consisting of cyclic tertiary aliphatic amines optionally dissolved in a non-aqueous solvent. The cyclic tertiary aliphatic amines are selected from the group consisting of azabicyclo[2.2.2]-octane (ABCO), and 1,4-diazabicyclo[2.2.2]-octane (DABCO).

Commercially available medical adhesives are available from Ethicon as Dermabond™ Advanced™ and Dermabond™ Prineo™.

### SUMMARY OF THE INVENTION

In view of the above, the art still needs medical adhesives which overcome the problems and issues noted above, as well as others. The problems are solved by the medical adhesive compositions of the present invention, which have relatively low extractables or migration of components out of or into the adhesive composition, thereby increasing patient safety. Reduction of formaldehyde migration out of a medical adhesive is advantageous for patients. Still further, the manner in which the medical adhesive compositions of the invention polymerize or cure inherently results in a relatively low acute exotherm, also increasing patient safety.

The medical adhesive compositions of the invention are based on the synergistic combination of an alkyl cyanoacrylate and a polymer modifier which at least imparts one or more of properties of flexibility and crosslinking to the compositions. The polymer modifier is reactive with the alkyl cyanoacrylate through functional groups thereof. The polymer modifier preferably has a low Tg and is miscible with the alkyl cyanoacrylate. In a preferred embodiment the polymer modifier comprises an elastomer having one or more cyanoacrylate groups, with the elastomer being one or more of a silicone elastomer, polyurethane, and polyisobutyrate.

In preferred embodiments, the medical adhesive compositions of the invention are relatively high viscosity systems for example having a viscosity from about 5 to about 10,000 mPa·s, about 20 mPa·s to about 5000 mPa·s, or about 80 to about 600 mPa·s as measured by a cone and plate viscometer (ASTM D4287-00(2019)).

In a preferred embodiment, the polymer modifier has a relatively high molecular weight which contributes to or is otherwise not detrimental to desirable bond strength. In order to specifically tailor properties of the adhesive composition, one or more of the following components can optionally be included: a rheology modifier, a stabilizer such as a free-radical inhibitor and/or anionic inhibitor, a polymerization activator, a medicament, and colorant.

In some preferred embodiments, medical tissue adhesive compositions are provided including a rheology modifier which advantageously provides a system having a desirable viscosity while still providing strong bond strength, even though the rheology modifier does not contribute to the latter. Such a medical adhesive system was difficult to develop and not known in the art, which instead in one embodiment utilized, as described in the Background of the Invention, relatively low molecular weight cyanoacrylate tri-telechelic star polymers having polyisobutylene chains terminated with cyanoacrylate groups (High-Ø(PIB-CA)3); 2-octyl cyanoacrylate (Oct-CA); and a relatively low molecular weight (Mn=1,000-4,000 g/mol) cyanoacrylate tri-telechelic star polymer having polyisobutylene chains terminated with cyanoacrylate groups (Low-Ø(PIB-CA)3) as a solubilizer.

Cured compositions of the adhesive composition are generally in the form of a coating, film, or layer, all said terms being interchangeably used herein.

The cured compositions exhibit a desirable moisture vapor transmission rate as well as a desirable oxygen permeability rate.

In order to reduce extractables in the cured composition, in one preferred embodiment the composition is free of a plasticizer.

In a further important aspect of the invention, an adhesive composition kit is provided that can be utilized to apply the adhesive to a wound, incision, or other desired tissue surface of a patient or animal. In a preferred embodiment the kit includes an applicator which comprises a first container such as an ampule and an initiator component. The first container comprises the adhesive composition described herein. In additional embodiments, the adhesive composition kit includes an adherable substrate such as a mesh, fabric, non-woven, sponge or porous material (e.g., a foam pad). The adherable substrate can be first applied to an incision or wound and the adhesive composition is thereafter applied over the outer surface thereof.

In an important aspect of the present invention, the adhesive composition kit is provided in a sterile, sealed package comprising the applicator, first container with the adhesive composition therein and optionally the adherable substrate. Providing the sterile applicator and adherable substrate in a sealed package reduces the risk of the patient encountering harmful organisms therefrom.

In a first embodiment a tissue adhesive composition is disclosed, comprising
at least one alkyl cyanoacrylate, wherein the alkyl group has from 1 to 20 carbon atoms and optionally including heteroatoms; and
a polymer modifier having one or more cyanoacrylate groups,
wherein the polymer modifier comprises one or more of a silicone elastomer, polyurethane and polyisobutyrate,
wherein i) the polymer modifier is present in an amount from about 3 wt. % to about 60 wt. % based on the total weight of the composition, and/or
   ii) the double bonds of the polymer modifier forming crosslinks are present in the range generally from 0.25 mol % to 40 mol % or desirably from 0.50 mol % to 20 mol % or preferably from 2 mol % to 10 mol % with respect to total polymerizable double bonds present in the composition; and
wherein at least some of the polymer modifier copolymerizes with the at least one alkyl cyanoacrylate upon polymerization of the adhesive composition.

In a second embodiment according to the first embodiment, the polymer modifier has two or more cyanoacrylate groups.

In a third embodiment according to any of the first and second embodiments, a plurality of polymer modifiers are present having different numbers of cyanoacrylate groups.

In a fourth embodiment according to any of the first through third embodiments, the composition has a migration out of the composition of formaldehyde that is less than formaldehyde migration out of the composition without the polymer modifier present measured utilizing hydrolytic degradation following the procedure set forth in Tissue Adhesive for the Topical Approximation of Skin-Class II Special Controls Guidance for Industry and FDA Staff May 30-2008, for >=7 days.

In a fifth embodiment according to any of the first through fourth embodiments the polymer modifier has a weight average molecular weight that ranges from about 250 g/mol to about 10,000 g/mol, desirably from about 1,000 g/mol to about 8,000 g/mol and preferably from about 1,500 g/mol to about 5,000 g/mol weight average as measured according to size exclusion or gel permeation chromatography or through determination of IV by viscosity, wherein the adhesive composition prior to polymerization has a viscosity that ranges from about 5 to about 10,000 mPa·s, desirably from about 20 to about 5,000 mPa·s, and preferably from about 80 to about 600 mPa·s as measured by cone and plate viscometer (ASTM D4287-00(2019)).

In a sixth embodiment according to any of the first through fifth embodiments, the silicone elastomer comprises polydimethylsiloxane, and wherein the alkyl cyanoacrylate is one or more of butyl cyanoacrylate and 2-octyl cyanoacrylate.

In a seventh embodiment according to any of the first through sixth embodiments, the composition includes a plasticizer or is free of a plasticizer.

In an eight embodiment according to any of the first through seventh embodiments, further including one or more of an anionic stabilizer, a free radical stabilizer, a rheology modifier, a medicament, and a color.

In a ninth embodiment according to any of the first through eight embodiments, further including an initiator, and wherein the initiator is present in an amount that ranges from about 0.01 wt. % to about 1 wt. %.

In a tenth embodiment a tissue adhesive composition is disclosed, comprising an applicator comprising a first container and an initiator component,
the first container comprising a topical adhesive composition, comprising: at least one alkyl cyanoacrylate, wherein the alkyl group has from 1 to 20 carbon atoms; and
a polymer modifier having one or more cyanoacrylate groups,
wherein the polymer modifier comprises one or more of a silicone elastomer, polyurethane and polyisobutyrate,
wherein i) the polymer modifier is present in an amount from about 6 wt. % to about 60 wt. % based on the total weight of the composition and/or
   ii) the double bonds of the polymer modifier forming crosslinks are present in the range generally from 0.25 mol % to 40 mol % or desirably from 0.50 mol % to 20 mol % or preferably from 2 mol % to 10 mol % with respect to total polymerizable double bonds present in the composition; and
wherein at least some of the polymer modifier copolymerizes with the at least one alkyl cyanoacrylate upon polymerization of the adhesive composition.

In an eleventh embodiment according to the tenth embodiment, the adhesive composition is formulated according to any of the second through nineth embodiments.

In a twelfth embodiment according to any of the twelfth through thirteenth embodiments, the kit includes a sealed package, comprising the applicator therein wherein the applicator and contents thereof are sterilized.

In a thirteenth embodiment according to any of the tenth through twelfth embodiments, the applicator is sterilized to provide a sterility assurance level (SAL) of at least 10⁻³ or 10⁻⁶, 10⁻¹² as measured according to an appropriate validation method.

In a fourteenth embodiment a method for preparing a kit suitable for applying a tissue adhesive is disclosed, comprising the steps of:
obtaining the tissue adhesive composition according to any of the first through night embodiments;
adding the tissue adhesive composition to a container;
sealing the container;
placing the sealed container in an applicator;
sterilizing the applicator and contents thereof;
sealing the sterilized applicator within a kit container.

For the avoidance of doubt, it is understood that while various embodiments or aspects of the invention are described individually, it should be clear that two or more embodiments or aspects can be, and often times are, present in a single device, method and/or kit according to the present invention.

### BRIEF DESCRIPTION OF THE DRAWINGS

The invention will be better understood, and other features and advantages will become apparent by reading the detailed description of the invention, taken together with the drawings, wherein:

FIG. 1 is a perspective view of one embodiment of an applicator including an ampule of the adhesive composition of the present invention; and

FIG. 2 is a perspective view of one embodiment of an adhesive composition kit of the present invention including an applicator including a first container of an adhesive composition in a polymerization activator. The kit also includes an adherable substrate such as mesh.

### DETAILED DESCRIPTION OF THE INVENTION

Medical adhesive compositions are described herein, which are well suited for use as a closure for wounds or surgical incisions. The adhesive composition is applied to living tissue as a liquid of desired viscosity which thereafter polymerizes or cures, forming a coating, film, or layer upon the tissue to which it has been applied. As utilized herein the terms “polymerize” and “cure” and variations thereof are used to describe the reaction the liquid adhesive composition undergoes upon application to tissue to form a film, layer, or other coating.

In some embodiments, an adherable substrate such as a polymer or other mesh is utilized in combination with the adhesive. In such cases, the adherable substrate is first applied in the area of the wound, incision or the like and the adhesive composition is applied over the adherable substrate and optionally to part of the surrounding tissue adjacent thereto. The adhesive composition will penetrate the mesh, due to the open pores thereof. Upon curing, a composite is formed from the adhesive composition and substrate in order to provide a protective layer for the patient against harmful organisms that can cause infection.

### Alkyl Cyanoacrylate

The adhesive composition according to the present invention comprises at least one alkyl cyanoacrylate that undergoes polymerization in order to form the cured coating, film, or layer. The alkyl group of the alkyl cyanoacrylate generally has from 1 to 20 carbon atoms, desirably from 2 to 16 carbon atoms and preferably from about 6 to about 10 carbon atoms. It is to be understood that the alkyl cyanoacrylates encompass compounds that contain heteroatoms in the “alkyl” side chain. Mixtures of different alkyl cyanoacrylate monomers can be utilized in the adhesive composition having different numbers of alkyl groups selected from the ranges above.

Alkyl cyanoacrylates having relatively low numbers of carbon atoms tend to polymerize quickly, especially upon exposure to traces of moisture on surfaces or tissue such as skin. The cyanoacrylate group in such compounds is highly reactive towards nucleophiles because of the presence of the two electron withdrawing substituents, namely the nitrile or cyano group (CN—) and the carboxylate ion (COO—). For example, lower alkyl cyanoacrylates such as methyl cyanoacrylate and ethyl cyanoacrylate polymerize substantially instantly in the presence of surface moisture, via anionic polymerization routes in the absence of inhibitors. Rates of polymerization are slower with higher alkyl cyanoacrylates, due at least in part to the lower molar concentrations of the cyanoacrylate groups. Desirable polymerization rates can be obtained by utilizing cyanoacrylates with different numbers of carbon atoms in the alkyl group and/or supplementing the adhesive composition with a polymerization activator.

The alkyl cyanoacrylate monomer enables the adhesive to bond to tissue surfaces, such as skin through the polymerization reaction of the adhesive monomer.

In a preferred embodiment, the alkyl cyanoacrylate is present in the adhesive composition in an amount from about 10 wt. % to about 90 wt. %, and preferably from about 50 wt. % to about 80 wt. % based on the total weight of the adhesive composition.

### Polymer Modifier

The adhesive compositions of the present invention also include a polymer modifier having one or more cyanoacrylate groups. The polymer modifier is added to the adhesive composition to alter or enhance its properties and/or performance.

Advantageously, the polymer modifier has been discovered to reduce formaldehyde production and/or migration out of the polymerized adhesive. Still further, the polymer modifier has been found to reduce the tack of the polymerized adhesive, as compared to a composition free of the modifier. In one embodiment, the polymer modifier can function as a flexibility modifier in order to enhance the resulting polymer film's pliability. In additional embodiments, the polymer modifier can function as a hydrophobicity modifier which can prevent or reduce water ingress into the adhesive compositions and decrease formaldehyde formation. The flexibility modifier thus aids the film in conforming to the surface of the skin and also withstand bending and stretching. In addition, the polymer modifier increases the life of the film due to the durability it imparts to the system. Still further, the polymer modifier contributes to film life by contributing to the ability to keep the film intact and bonded to the skin for a sufficient time to allow for wound or incision healing.

While not wishing to be bound by theory, in some embodiments it is believed that the polymer modifier forms bonds between the polymerized strands of polymerized alkyl cyanoacrylate, and thus acts as a cross-linking agent. The formation of a cross-linked polymerized network enhances the ability of the film formed from the adhesive composition to remain intact once bonded to the skin or other tissue by enabling the polymer film to bend with the flexing forces normally exerted on the skin or other tissue surface without breaking. The cyanoacrylate group present in the polymer modifier may also, in some embodiments, also adhere or attach to living tissue. A variety of forces which could also serve to embed the polymer modifier in the polymer network and decrease migration include, but are not limited to, van der Waals forces, stearic hindrance, hydrogen bonding, covalent bonding, and ligation, etc.

The polymer film flexibility and conformance to the skin or tissue surface provided through use of the polymer modifier in combination with the alkyl cyanoacrylate beneficially maintains skin or tissue edge apposition and allows normal healing of a wound or incision.

The polymer modifier includes one or more cyanoacrylate groups that are able to react with the alkyl cyanoacrylate component included in the adhesive composition. Examples of suitable polymer modifiers include, but are not limited to, silicone elastomers, polyurethanes and polyisobutyrate, each of which have one or more cyanoacrylate groups, and preferably two or more cyanoacrylate groups.

Silicone elastomer when utilized as a polymer modifier provides oxygen and moisture permeability, otherwise known as breathability. Breathability allows for the evaporation of water vapor through the film which thereby promotes healing of the wound or incision. As mentioned above, silicone elastomer also contributes elasticity to the film formed from the adhesive composition, as well as good mechanical properties such as tensile strength and elongation. Silicone elastomers also have biocompatibility with living tissues and are substantially optically transparent, the latter allowing inspection of a wound or incision.

In preferred embodiments, the silicone elastomer which includes the at least one cyanoacrylate group is based on one or more of polydimethylsiloxane (PDMS), methylhydrosiloxane-dimethylsiloxane diblock copolymer, a methylhydrosiloxane homopolymer, and a methylhydrosiloxane-dimethylsiloxane random copolymer having a cyanoacrylate-based substituent replacing the hydrogen attached to the Si within the methylhydrosiloxane moiety.

In some embodiments, the at least one cyanoacrylate group is a —O—CO—C(CN)═CH₂ group. In one or more of these embodiments, the at least one cyanoacrylate group may be directly attached at a terminal end of the silicone elastomer. This is particularly true where the silicone elastomer is polydimethylsiloxane. In other embodiments, the at least one cyanoacrylate group is directly attached to the interior backbone of the silicone elastomer. This is particularly true where the cyanoacrylate group is attached to the methylhydrosiloxane-substituted moiety within the methylhydrosiloxane homopolymers, methylhydrosiloxane-dimethylsiloxane diblock copolymer or methylhydrosiloxane-dimethylsiloxane random copolymer.

In some embodiments, the silicone elastomer is functionalized with at least two cyanoacrylate groups. In some embodiments, the cyanoacrylate groups are attached to both or all terminal ends of a silicone elastomer. In other embodiments, the cyanoacrylate groups are attached to only the interior backbone of the silicone elastomer. Still in other embodiments, one or more of the cyanoacrylate groups are attached to one or more of the terminal ends of the silicone elastomer, while one or more other cyanoacrylate groups are attached to one or more places within the backbone of the silicone elastomer.

In additional embodiments, the silicone elastomer has a spacer group between the silicone elastomer and at least one of the cyanoacrylate groups. In one or more of these embodiments, the spacer group is attached to a terminal end of the silicone elastomer and the cyanoacrylate group is attached to the spacer group. In other embodiments, the spacer group is attached to the interior backbone of the silicone elastomer and the cyanoacrylate group is attached to the spacer group. In one or more of these embodiments, the spacer group is a polyalkyl block. In one or more other embodiments, the spacer group is a polyalkylene oxide block. Where the spacer group is a polyalkyl block or a polyalkylene oxide block, the spacer group may have from 1 to 16 carbon atoms. In one embodiment, the spacer group is an allylic block. In one or more embodiments, the spacer group and cyanoacrylate group (CA) provide for a —CH₂—CH₂—CH₂—CA substituent attached to the silicone elastomer.

In further embodiments, the polymer modifier is a polyurethane which has been modified to include one or more cyanoacrylate groups. Polyurethanes can be formed from one or more of polyether and polyester polyols. Following are non-limited examples of suitable polyols and approximate ranges for the Tg of polyurethanes based on different types of polyols:

### Polyether Polyols:

Polypropylene glycol (PPG): Typically around −50° C. to −60° C.
Polyethylene glycol (PEG): Typically lower than −60° C.
Poly(tetramethylene ether) glycol (PTMEG): Typically around −55° C. to −65° C.
Polycaprolactone polyols: Typically around −60° C. to −70° C.

### Polyester Polyols:

Polycarbonate polyols: Typically around −40° C. to −50° C.
Polybutylene adipate (PBA): Typically around −45° C. to −55° C.
Polyoxyethylene adipate (POEA): Typically around −40° C. to −50° C.
Polyethylene adipate (PEA): Typically around −45° C. to −55° C.

In additional embodiments, the polymer modifier is polyisobuytrate which has been modified to include one or more cyanoacrylate groups.

In an important aspect of the present invention, it has been discovered that the polymer modifier aids in reducing formaldehyde emission from the adhesive composition. The adhesive compositions have a migration out of the composition of formaldehyde that is less than the formaldehyde migration out of a comparative composition without the polymer modifier present, as measured utilizing hydrolytic degradation. The advantage is notable as formaldehyde is a known environmental hazard. In additional embodiments, the polymer modifier also aids in preventing migration of potential environmental degradation agents into the polymerized adhesive composition. Still further, the polymer modifier can prevent migration of other components out of the adhesive composition upon polymerization.

The polymer modifier preferably has a molecular weight that ranges from about 250 g/mol to about 10,000 g/mol, desirably from about 1,000 g/mol to about 8,000 g/mol and preferably from about 1,500 g/mol to about 5,000 g/mol weight average as measured according to size exclusion or gel permeation chromatography or through determination of IV by viscosity.

The polymer modifier having one or more cyanoacrylate groups is present in an amount generally from about 0.5 wt. % to 85 wt. %, desirably from about 2 wt. % to about 60 wt. %, and preferably from about 5 wt. % to about 45 wt. %, most preferably from about 15 wt. % to about 35% based on the total weight of the composition, as set forth herein, wherein the composition at least includes the at least one alkyl cyanoacrylate, and optionally other components such as described herein.

In a further embodiment, the double bonds of the polymer modifier forming crosslinks are present in the range generally from 0.25 mol % to 40 mol % or desirably from 0.50 mol % to 20 mol % or preferably from 2 mol % to 10 mol % with respect to total polymerizable double bonds present in the composition.

The polymer modifier may also be defined with reference to its glass transition temperature, Tg. In some preferred embodiments, the polymer modifier has a relatively low Tg that ranges generally from about −80° C. to about 30° C.

### Rheology Modifier

A rheology modifier is optionally present in the adhesive compositions of the present invention. The rheology modifier is employed in the adhesive composition to enhance the user's ability to control the flow of the liquid adhesive during application to an intended surface, such as living tissue and the adherable substrate that can be present on the living tissue to be treated. The rheology modifier either increases or decreases the viscosity of an adhesive composition including the at least one alkyl cyanoacrylate and polymer modifier having one or more cyanoacrylate groups. Suitable amounts of the rheology modifier are utilized in order to prevent the liquid from flowing or migrating away from the intended application site as the adhesive composition is spread over the desired surface, preferably utilizing an applicator. Further considerations regarding the amount of rheology modifier include the necessity that the adhesive compositions remain soluble prior to and during application prior to polymerizing homogeneously after application.

Rheology modifying agents suitable for use in the invention should be compatible with the alkyl cyanoacrylate and polymer modifier utilized. The rheology modifiers should also be compatible with the living tissue to which they are applied as part the adhesive composition. Preferably, the rheology modifiers are miscible in adhesive composition at room temperature.

In some embodiments, the rheology modifiers include one or more (co)polymers comprising structural units derived from (meth)acrylate monomers. As used herein, the term “(meth)acrylate” refers to either acrylate or methacrylate and the term “(co)polymer” refers to either a polymer or copolymer. Suitable (meth)acrylate monomers include, but are not limited to, alkyl (meth)acrylates wherein the alkyl group ranges from 1 to about 20 carbon atoms, for example methyl (meth)acrylate, ethyl (meth)acrylate, butyl (meth)acrylate, pentyl (meth)acylate, hexyl (meth)acrylate, heptyl (meth)acrylate, 2-ethyl hexyl (meth)acrylate, octyl (meth)acrylate, cyclohexyl (meth)acrylate, phenyl (meth)acrylate, benzyl (meth)acrylate and 2-phenylethyl (meth)acrylate.

Examples of suitable comonomers include alkyl cyanoacrylates, wherein the alkyl group has from 1 to 20 carbon atoms and optionally includes heteroatoms, such as methyl cyanoacrylate, ethyl cyanoacrylate, propyl cyanoacrylate, iso-propyl cyanoacrylate, butyl cyanoacrylate, n-butyl cyanoacrylate, neopentyl cyanoacrylate, octyl cyanoacrylate, ethylhexyl cyanoacrylate, beta-methoxyethyl cyanoacrylate, beta-ethoxyethyl cyanoacrylate, and octadecyl cyanoacrylate. Still other comonomers include phenylethyl cyanoacrylate, cyclohexenyl cyanoacrylate, tetrahydrofuran cyanoacrylate, trimethylsilane cyanoacrylate, and 1,6-hexanediol bis-cyanoacrylate.

Further non-limiting examples of rheology modifiers include, but are not limited to, polycaprolactone, copolymers of alkylacrylate and vinyl acetate, lactic-glycolic acid copolymers, lactic acid-caprolactone copolymers, polyorthoesters, copolymers of alkyl methacrylates and butadiene, polyoxalates, and triblock copolymers of polyoxypropylene flanked by two hydrophilic chains of polyoxyethylene.

Still other rheology modifiers are well known in the art. See for example Robello et al., Degradation and Stabilization of Polycyanoacrylates; Duffy et al., Radial Polymerization of Alkyl 2-Cyanoacrylates; Han et al., Controlled degradation of poly(ethyl cyanoacrylate-c-methyl methacrylate) (PECA-co-PMMA) copolymers; Han et al., Synthesis and degradation behavior of poly(ethyl cyanoacrylate); Ryan et al., Novel sub-ceiling temperature rapid depolymerization-repolymerization reactions of cyanoacrylate polymers; and Leonard et al., Synthesis and Degradation of Poly(alkyl α-Cyanoacrylates), all herein fully incorporated by reference.

Mixtures of different rheology modifying agents can be utilized if desired.

The rheology modifying agents when present, are utilized in an amount generally from about 1 wt. % to about 25 wt. %, desirably from about 2 wt. % to about 20 wt. % and preferably from about 5 wt. % to about 15 wt. %, based on the total weight of the adhesive composition.

### Stabilizers

In some embodiments, the adhesive composition includes one or more stabilizers that function to prevent unintended polymerization of the primary adhesive components, namely the at least one alkyl cyanoacrylate and polymer modifier having one or more cyanoacrylate groups. Suitable stabilizers include anionic stabilizers or inhibitors and free radical stabilizers or inhibitors.

Anionic stabilizers, when present, react with and neutralize any excess hydroxyl ions (⁻OH) that may be present in the adhesive composition. Hydroxyl ions may result from the presence of water and could otherwise initiate the polymerization reaction if such anionic stabilizers were not present. Examples of anionic stabilizers include, but are not limited to, sulfur dioxide, sulphonic acids, toluenesulfonic acid, fluorosulfonic acid, sultones, sulfuric acid, sulfur trioxide, phosphorous acid, mercaptans, alkyl sulfoxides, alkyl sulfides, lactone, alkyl sulfates, alkyl sulfites, 3-sulfolene, alkyl sulfones, picric acid, boron trifluoride, BF₃-ether complexes, citric acid, tin (IV) chloride, iron (III) chloride, acid chelates perchloric acid, hydrochloric acid, hydrobromic acid, trichloroacetic acid and weak acids such as phosphoric acid, acetic acid, hydrofluoric acid, formic acid, benzoic acid, oxalic acid, hydrofluoric acid, nitrous acid, sulfurous acid, and other organic acids, for example C₂ to C₆ organic acids, and mixtures of two or more thereof.

The anionic stabilizers, when present, are used in a total amount generally from an amount of about 2 ppm to about 500 ppm, and preferably about 10 ppm to about 200 ppm based on the total weight of the adhesive composition.

Free radical stabilizers, when present, react with and neutralize free radicals that may be present in the adhesive composition. Free radicals are unstable molecular species that have one or more unpaired electrons. The unpaired electrons allow the free radicals to participate or otherwise initiate a polymerization reaction of one or more of the alkyl cyanoacrylate and polymer modifier.

Free radical stabilizers are known in the art. Nonlimiting examples of free radical stabilizers include, but are not limited to, hydroquinone, methyl hydroquinone, hydroquinone monomethyl ether, t-butyl hydroquinone, phenothiazine, catechol, pyrogallol, benzoquinone, 2-hydroxybenzoquinone, p-methoxy phenol, 1-butyl catechol, butylated hydroxytoluene, butylated hydroxyanisole (BHA), butyl hydroxy toluene (BHT), catechol, 4-tert-butoxyphenol, 4-ethoxyphenol, 3-methoxyphenol, 4-methoxyphenol (MP), 2-tert-butyl-4-methoxyphenol, and 2,2-methylene-bis-(4-methyl-6-tert-butylphenol). Hydroquinone is a preferred free radical stabilizer in one embodiment of the present invention.

The free radical stabilizers, when present, are used in a total amount generally from an amount from about 200 ppm to about 15000 ppm, desirably about 1000 ppm to about 10000 ppm, and preferably 2000 ppm to 8000 ppm based on the total weight of the adhesive composition.

### Plasticizer

In some embodiments, the adhesive compositions are free of a plasticizer. In other embodiments, the adhesive composition includes a plasticizer. Plasticizers are generally low molecular weight components that reduce the glass transition temperature of the polymer matrix. Examples of suitable plasticizers include, but are not limited to citrates such as dibutyl citrate, dibutyl acetyl citrate and triethyl citrate; sebacates such as dioctyl sebacate; glycols such as polyethylene glycol and propylene glycol; glycerol and glycerol derivates such as mono-, di- or triacetates of glycerol.

### Optional Components

In various embodiments, the adhesive compositions according to the invention can include other optional components depending on the end use application. For example, the adhesive compositions can include one or more of a medicament and a colorant, or a low boiling point solvent. It should be clear to one of ordinary skill in the art that an optional component may have more than one function. For example, the low boiling point solvent may also function as a colorant.

### Initiator

While polymerization of the adhesive composition can be initiated by moisture on or from tissue to which the adhesive composition is applied, such as skin of a patient, in some embodiments it is desirable to utilize an initiator to control the polymerization rate. As combining an initiator with the reactants of the adhesive composition including the at least one alkyl cyanoacrylate and polymer modifier causes the composition to polymerize, the initiator is not mixed with at least the reactable components until immediately prior to application of the adhesive composition to desired tissue of a patient. A further function of the polymerization initiator is to help ensure consistent polymerization and film-forming during adhesive application. Some initiators may also be considered accelerants.

The polymerization reaction of the adhesive composition is initiated by a nucleophilic attack on the carbon-carbon double bond of a cyanoacrylate group, which creates an “electron sink” at the alpha-carbon that drives the reaction with adjacent monomer molecules to form a rapidly growing chain or (co)polymer.

Suitable initiators for the adhesive compositions of the present invention include, but are not limited to, water; bases; anions, such as strong anion exchange resins; alcohols such as methanol; amines such as tertiary amines; phosphines calixarenes; oxacalixarenes; silacrowns; crown ethers; cyclodextrin and its derivatives; polyethers; aliphatic alcohol; various aliphatic carboxylic acid esters; benzoyl peroxide; phosphonium salts and ammonium salts, such as alkyl ammonium salts and quaternary ammonium salts. In additional embodiments other initiators well known to the art can be utilized.

Examples of suitable amine compounds include triethyl amine, diethyl amine, butyl amine, isopropyl amine, tributyl amine, N,N,-dimethyl aniline, N,N-diethyl aniline, N, N-dimethyl-p-toluidine, N,N-dimethyl-m-toluidine, N, N-dimethyl-o-toluidine, dimethyl benzyl amine, pyridine, picoline, vinyl pyridine, ethanolamine, propanolamine and ethylene diamine,

The quaternary ammonium salt may be any of a group of ammonium salts in which organic radicals have been substituted for all four hydrogens of the original ammonium cation. In embodiments, the quaternary ammonium salt may have the general formula A:

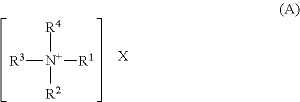

wherein R¹, R², R³ and R⁴ are each, independently, a substituted or unsubstituted straight, branched or cyclic alkyl group; a substituted or unsubstituted aromatic ring; or a substituted or unsubstituted aralkyl group, wherein the alkyl groups, aromatic rings or aralkyl groups may optionally further contain heteroatoms such as O, N, and S; and X is an anion such as a halide, for example chloride, bromide, or fluoride, or hydroxyl. In embodiments, R¹, R², R³ and R⁴ are Ci-Cs alkyl groups, preferably, C₁-C₄ alkyl groups, or an aralkyl group. By way of example, quaternary ammonium salts may include, but are not limited to, tetrabutylammonium fluoride, tetramethylammonium fluoride, tetraethylammonium fluoride, tetraoctylammonium fluoride, benzyltrimethyl ammonium fluoride, domiphen bromide, butyrylcholine chloride, benzalkonium bromide, benzalkonium chloride, acetyl choline chloride, or a combination thereof. In some embodiments the preferred initiator is benzalkonium chloride (BAC).

Still other initiators include, but are not limited to, N,N-dimethyl-p-toluidine (DMT), azabicyclo[2.2.2]-octane (ABCO), and 1,4-diazabicyclo[2.2.2]-octane (DABCO).

The amount of initiator utilized in the adhesive composition depends on factors such as the type of alkyl cyanoacrylate monomers utilized, the type of polymer modifier and amounts of other components such as the anionic stabilizer, free radical stabilizer, viscosity modifier, and the desired rate of polymerization. However, generally, the amount of initiator can vary from about 0.01 wt. % to about 1 wt. %, desirably from about 0.05 wt. % to about 0.95 wt. %, and preferably from about 0.10 wt. % to about 0.90 wt. %, based on the total weight of the adhesive composition.

### Adhesive Composition Viscosity

The tissue adhesive composition of the invention including at least one alkyl cyanoacrylate and polymer modifier having one or more cyanoacrylate groups is formulated to have a desirably viscosity that allows facile application to a desired surface. If the viscosity of the adhesive composition is too low, it may be spread to unintended surfaces upon application and/or the resulting film may not have sufficient thickness and/or strength. If the adhesive composition viscosity is too high, it may be one or more of difficult to apply via an applicator and form a film that is too thick.

In view of the above, the viscosity of the adhesive composition prior to polymerization has a viscosity that ranges from about 5 to about 10,000 mPa·s, desirably from about 20 to about 5,000 mPa·s, and preferably from about 80 to about 600 mPa·s as measured by cone and plate viscometer (ASTM D4287-00(2019)).

### Tissue Adhesive Composition Kit

In preferred embodiments of the present invention, the adhesive compositions of the present invention are provided in a tissue adhesive composition kit that can be utilized by a health care professional to treat a patient, animal, etc. Advantageously, the kit can be provided in a sterile form which reduces the risk of the patient encountering harmful organisms. The kit includes an applicator for dispensing and/or applying the fluid adhesive composition on the desired tissue surface of the patient, for example a wound or surgical incision in order to adhere or hold together separated tissue, or even intact skin if desired. The applicator is particularly suitable for medical use as a soft tissue, or more precisely, a topical adhesive, but could be used for other applications.

The applicator comprises a first container such as an ampoule, and an initiator component. The first container comprises an adhesive composition of the present invention. In additional embodiments, the adhesive composition kit includes an adjunct would closure device, for example an adherable substrate such as a mesh, fabric, non-woven, sponge or porous material (e.g. a foam pad). The adherable substrate can be applied to tissue, such as a wound or incision in a first step, and the adherable composition thereafter applied over the outer surface of the adherable substrate, whereupon polymerization of the adhesive composition produces a composite including a polymerized adhesive composition and adherable substrate.

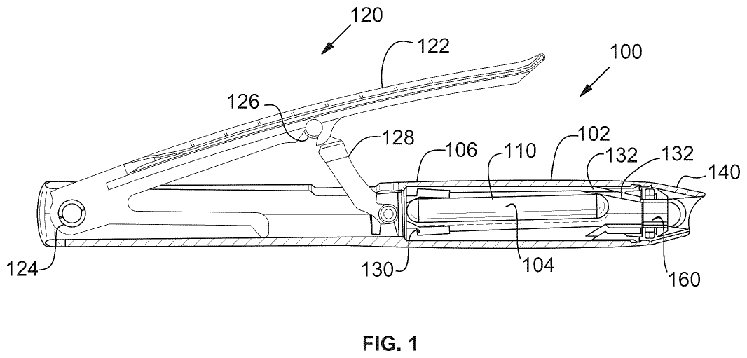

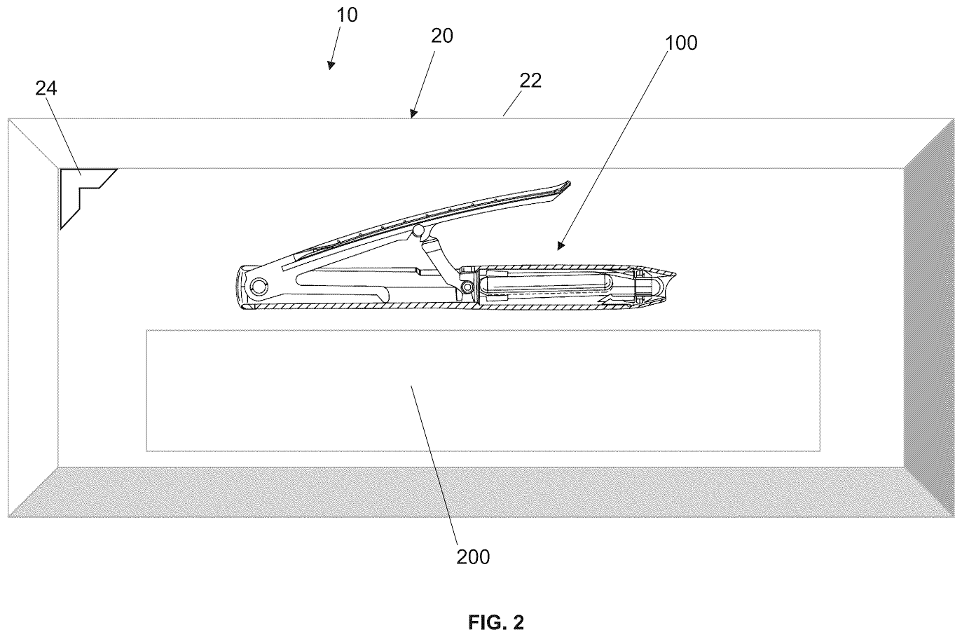

Referring now to the drawings, wherein the reference numerals represent the same or similar parts throughout the several views, FIG. 1 illustrates an applicator 100 including a first container 110 including the adhesive composition of the present invention. FIG. 2 illustrates kit 10 including a housing 20 having sealed therein applicator 100 and an adherable substrate 200. Housing 20 generally includes a tray 22 or other container that is sealed for example utilizing removable film 24, which can be peeled away from the tray to reveal the sterile contents of the kit 10, when necessary. The film 24 is partially shown in FIG. 2.

Applicator 100 facilitates easy and comfortable application of a desired quantity of the adhesive composition, in particular in a pen-like housing 102. In preferred embodiments, a desired amount of polymerizable adhesive composition 104 is prepackaged in the applicator in a first container, such as frangible ampoule 110 that is broken upon activation of the applicator. The ampoule is preferably a glass, e.g., borosilicate, metal, e.g. aluminum, or other material that is shaped like a suppository that is illustrated as cylindrical with rounded end members which may or may not be symmetrical and have the same shape.

Applicator 100 has a longitudinal axis and an actuator 120 having a handle 122 with a boss 124 captured in a recess of the body so that the handle pivots, causing a lever linkage 128 having a pivot 126 which applies a force relative to the body 106 portion in the direction of the longitudinal axis. Movement of handle 122 towards the body 106 causes plunger 130 to move ampoule towards the lower end of the body where it encounters cracking member 132 which breaks the ampoule 110, allowing the adhesive composition to flow out therefrom. Delivery tip 140 of applicator 100 includes porous filter 160 which includes initiator embedded or otherwise contained thereon. As the adhesive composition 104 flows out of tip 140, it passes by or otherwise through porous filter 160 initiating polymerization of the adhesive composition.

It is to be understood that while one embodiment of an applicator is described herein, other embodiments may be suitable. For example, please see U.S. Pub. 2023/0233197 A1, herein fully incorporated by reference.

### Sterilization

In a preferred embodiment, the adhesive composition is sterilized via a suitable method in order to provide a sterility assurance level (SAL) of at least 10⁻³, or 10⁻⁶, or 10⁻¹² as measured according to an appropriate validation method. When the adhesive composition prior to polymerization has a viscosity of less than about 600 mPa·s, sterilization can be performed through filtration. Still further, the adhesive composition can be sterilized through use of radiation such as from an electron beam or gamma radiation. Other sterilization methods include steam or dry heat. Such sterilization methods are well known to those of ordinary skill in the art.

In a further preferred embodiment, the applicator containing an ampoule of the adhesive composition is sterilized, thereby providing the sterility assurance level defined above. The applicator and components thereof as well as the ampoule can be sterilized using gases such as ethylene oxide, ozone, hydrogen dioxide, vapor phase hydrogen peroxide, and plasma, etc. An appropriate validation method is selected in order to demonstrate that the sterilization process delivers the desired sterility assurance level, for example ANSI/AAMI/ISO 11137:2006 for radiation, ANSI/AAMI/ISO 11135 for gas sterilization and AAMI/ISO Technical Information Report (TIR) 13409 for radiation.

### EXAMPLES

The examples set forth below are provided to illustrate the features of the compositions of the present invention and are not intended to limit the scope of the invention.

The following raw materials were utilized for the examples.

| | |
|---|---|
| Alkyl | OCA: 2-octyl cyanoacrylate (Affinitica/ Bostik) |
| Cyanoacrylate | Prineo: Dermabond ® PrineoR-Johnson & |
| | Johnson Ethicon product (commercial blend) |
| | Cutiva: Cutiva ™-Resivant medical (future |
| | commercial blend including a silicone elastomer) |
| Polymer Modifier | Additive 1:Silicone elastomer with cyanoacrylate groups* |
| Plasticizer | Additive 2: Tributyl citrate [77-94-1] (Sigma Aldrich) |
| Anionic Stabilizer | Included in supplied products, no additional used |
| Free Radical | Included in supplied products, no additional used |
| Stabilizer | |
| Initiator | Present in Dermabond ® Prineo ® mesh where used |

*silicone produced according to: Q. Liu, H. Dong, S. Guo, Y. Zhang, E. Wang, Z. Qu, D. Chen, L. Huang, J.Hou, Y. Zheng, C. Wu. Nano Select. 2022, 3, 1557. doi.org/10.1002/nano.202200151 to a molecular weight of approximately 3,000 g/mol and an average functionality of 3 cyanoacrylates per molecule.

The following protocols were used for testing.

### Tack Test

Tack was measured by blotting cured films with cotton wool and estimating the surface coverage of cotton wool remaining on the test part-100% is full coverage, 0% is no residue. The test is based on a simplified version of the Cotton Ball print resistance test derived from ASTM D2064.

### Formaldehyde Test

The formaldehyde test was conducted on 1 drop of saline hydrolytic degradation solution. This drop was mixed with 10 μL of Quantofix HCHO-1 solution to result in an approximately 1:1 blend and a pH of 13-14. This test solution was immediately taken up onto a test trip (Quantofix® formaldehyde) and allowed to sit at room temperature for 1 min. The test strip was then compared to the color chart and a value of formaldehyde estimated in mg/L. Test kit: Quantofix® Formaldehyde, 10-200 mg/L HCHO, Macherey-Nagel, Duren, Germany.

### Hydrolytic Degradation

Hydrolytic degradation was performed on pre-weighed parts of cured (odorless) films. Each part/film (10-100 mg) was placed in a borosilicate vial (46×22.5 mm), sealed with a crimp cap (ND20 butyl/PTFE). 1 ml of saline solution (BD Posiflush SP 0.9% Sodium chloride, sterile) was added to each sample in the sealed vial. The vials were placed in a sand bath in a temperature-controlled oven at 50 C+/−2 C for 7 days. Once removed from the oven, samples were allowed to cool to room temperature before further testing. Hydrolytic degradation protocol is based on “Tissue Adhesive for the Topical Approximation of Skin—Class II Special Controls Guidance for Industry and FDA Staff”, May 30-2008, for greater than or equal to 7 days.

### Sample Preparation

Sample preparation: all formulations were blended at a relative humidity below 30% and sealed in vials. (Borosilicate (46×22.5 mm) with crimp cap (ND20 butyl/PTFE))

Formulations/blends were cured on microscope slides (sodalime glass) at 50° C.+/−5° C. on a hotplate in an open atmosphere until tacky (partially cured) then placed on an 8 mm glass plate in an oven at 50° C.+/−5° C. full of wet cloths (to increase humidity and finalize curing) for at least 1 hr or until odorless. These parts were allowed to sit in a clean environment at room temperature to equilibrate prior to sampling for hydrolytic degradation or evaluation for tackiness.

Samples prepared with mesh were coated onto glass slides onto which a piece of Dermabond® Prineo® mesh was then placed—this also accelerated cure due to the presence of activator in the mesh material as supplied.

The following compositions were prepared and tested as noted in the Table below.

| Reference | Base | Base | Additive | Additive |
|---|---|---|---|---|
| Number | Type | (wt %) | 1 (wt %) | 2 (wt %) |
| p1 | Prineo | 81.9% | 18.1% | 0.0% |
| p2 | Prineo | 83.9% | 16.1% | 0.0% |
| p3 | Prineo | 93.8% | 6.2% | 0.0% |
| o1 | OCA | 74.6% | 25.4% | 0.0% |
| o2 | OCA | 91.5% | 8.5% | 0.0% |
| o3 | OCA | 93.8% | 6.2% | 0.0% |
| o4 | OCA | 59.4% | 0.0% | 40.6% |
| o5 | OCA | 67.6% | 0.0% | 32.4% |
| o6 | OCA | 86.6% | 0.0% | 13.4% |
| t1 | Cutiva | 83.2% | 0.0% | 16.8% |
| t2 | Cutiva | 67.4% | 0.0% | 32.6% |
| t3 | Cutiva | 67.8% | 0.0% | 32.2% |

Table of Test Results

| | M if Mesh | Formaldehyde Test | Tack Test |
|---|---|---|---|
| Reference | used in | (Formaldehyde in | (Pick-up |
| Number | sample prep | ug/mg sample) | Value) |
| p1 | M | 0.04 | 0 |
| p2 | M | 0.45 | 2 |
| p3 | M | 0.77 | 5 |
| o1 | M | 0.16 | 2 |
| o2 | M | 0.09 | 0 |
| o3 | M | 0.46 | 2 |
| o4 | M | 1.60 | 100 |
| o5 | M | 1.43 | 100 |
| o6 | M | 0.67 | 50 |
| t1 | M | 0.10 | 0 |
| t2 | M | 0.28 | 5 |
| t3 | M | 0.46 | 10 |

Mesh weight is subtracted for analysis of formaldehyde concentration on weight sample/weight formaldehyde produced basis.

The test results clearly show the advantages of the invention, wherein the combination of a polymer modifier with an alkyl cyanoacrylate reduces formaldehyde migration as well as tack.

For the avoidance of doubt, the compositions and methods of the present invention encompass all possible combinations of the components, including various ranges of said components, disclosed herein. It is further noted that the term ‘comprising’ does not exclude the presence of other elements. However, it is also to be understood that a description of a product comprising certain components also discloses a product consisting of these components. Similarly, it is also to be understood that a description on a process comprising certain steps also discloses a process consisting of these steps.

In accordance with the patent statutes, the best mode and preferred embodiment have been set forth; the scope of the invention is not limited thereto, but rather by the scope of the attached claims.

What is claimed is:

1. A tissue adhesive composition, comprising:
at least one alkyl cyanoacrylate, wherein the alkyl group has from 1 to 20 carbon atoms and optionally including heteroatoms; and
a polymer modifier having one or more cyanoacrylate groups,
wherein the polymer modifier comprises one or more of a silicone elastomer, polyurethane and polyisobutyrate,
wherein i) the polymer modifier is present in an amount from about 3 wt. % to about 60 wt. % based on the total weight of the composition, and/or
ii) double bonds of the polymer modifier able to form a crosslinks are present in the range generally from 0.25 mol % to 40 mol % with respect to total polymerizable double bonds present in the composition; and
wherein at least some of the polymer modifier copolymerizes with the at least one alkyl cyanoacrylate upon polymerization of the adhesive composition.

2. The adhesive composition according to claim 1, wherein the polymer modifier has two or more cyanoacrylate groups.

3. The adhesive composition according to claim 1, wherein a plurality of the polymer modifiers are present having different numbers of cyanoacrylate groups.

4. The adhesive composition according to claim 1, wherein the composition has a migration out of the composition of formaldehyde that is less than formaldehyde migration out of the composition without the polymer modifier present measured utilizing hydrolytic degradation following the procedure set forth in Tissue Adhesive for the Topical Approximation of Skin-Class II Special Controls Guidance for Industry and FDA Staff May 30-2008, for >=7 days.

5. The adhesive composition according to claim 1, wherein the polymer modifier has a weight average molecular weight that ranges from about 250 g/mol to about 10,000 g/mol, desirably from about 1,000 g/mol weight average as measured according to size exclusion or gel permeation chromatography or through determination of IV by viscosity, and wherein the adhesive composition prior to polymerization has a viscosity that ranges from about 5 to about 10,000 mPa·s as measured by cone and plate viscometer (ASTM D4287-00(2019)).

6. The adhesive composition according to claim 1, wherein the silicone elastomer comprises polydimethylsiloxane, and wherein the alkyl cyanoacrylate is one or more of butyl cyanoacrylate and 2-octyl cyanoacrylate.

7. The adhesive composition according to claim 1, wherein the composition includes a plasticizer.

8. The adhesive composition according to claim 1, further including one or more of an anionic stabilizer, a free radical stabilizer, a rheology modifier, a medicament, and a colorant.

9. The adhesive composition according to claim 1, further including an initiator in an amount that ranges from about 0.01 wt. % to about 1 wt. % based on the total weight of the composition.

10. The adhesive composition according to claim 3, wherein the polymer modifier has a weight average molecular weight that ranges from about 1,000 g/mol to about 8,000 g/mol weight average as measured according to size exclusion or gel permeation chromatography or through determination of IV by viscosity, wherein the adhesive composition prior to polymerization has a viscosity that ranges from about 20 to about 5,000 mPa·s as measured by cone and plate viscometer (ASTM D4287-00(2019)).

11. The adhesive composition according to claim 10, wherein the silicone elastomer comprises polydimethylsiloxane, and wherein the alkyl cyanoacrylate is one or more of butyl cyanoacrylate and 2-octyl cyanoacrylate.

12. The adhesive composition according to claim 2, wherein the polymer modifier has a weight average molecular weight that ranges from about 1,500 g/mol to about 5,000 g/mol weight average as measured according to size exclusion or gel permeation chromatography or through determination of IV by viscosity, wherein the adhesive composition prior to polymerization has a viscosity that ranges from about 80 to about 600 mPa·s as measured by cone and plate viscometer (ASTM D4287-00(2019)).

13. The adhesive composition according to claim 12, wherein the silicone elastomer comprises polydimethylsiloxane, and wherein the alkyl cyanoacrylate is one or more of butyl cyanoacrylate and 2-octyl cyanoacrylate.

14. The adhesive composition according to claim 2, wherein a plurality of the polymer modifiers are present having different numbers of cyanoacrylate groups, wherein the composition has a migration out of the composition of formaldehyde that is less than formaldehyde migration out of the composition without the polymer modifier present measured utilizing hydrolytic degradation following the procedure set forth in Tissue Adhesive for the Topical Approximation of Skin—Class II Special Controls Guidance for Industry and FDA Staff May 30-2008, for >=7 days, wherein the composition includes a plasticizer, wherein the composition includes one or more of an anionic stabilizer, a free radical stabilizer, a rheology modifier, a medicament, and a colorant, and wherein the composition includes an initiator in an amount that ranges from about 0.01 wt. % to about 1 wt. % based on the total weight of the composition.

15. A tissue adhesive composition kit, comprising:
an applicator comprising a first container and an initiator component,
the first container comprising a topical adhesive composition, comprising:
at least one alkyl cyanoacrylate, wherein the alkyl group has from 1 to 20 carbon atoms; and
a polymer modifier having one or more cyanoacrylate groups,
wherein the polymer modifier comprises one or more of a silicone elastomer, polyurethane and polyisobutyrate,
wherein i) the polymer modifier is present in an amount from about 6 wt. % to about 60 wt. % based on the total weight of the composition and/or
ii) double bonds of the polymer modifier able to form crosslinks are present in the range generally from 0.25 mol % to 40 mol % with respect to total polymerizable double bonds present in the composition; and
wherein at least some of the polymer modifier copolymerizes with the at least one alkyl cyanoacrylate upon polymerization of the adhesive composition.

16. The tissue adhesive composition kit according to claim 15, wherein the polymer modifier has two or more cyanoacrylate groups, wherein the polymer modifier has a weight average molecular weight that ranges from about 250 g/mol to about 10,000 g/mol weight average as measured according to size exclusion or gel permeation chromatography or through determination of IV by viscosity, and wherein the adhesive composition prior to polymerization has a viscosity that ranges from about 5 to about 10,000 mPa·s as measured by cone and plate viscometer (ASTM D4287-00(2019)).

17. The tissue adhesive composition kit according to any of claim 16, wherein the kit includes a sealed package, comprising the applicator therein, wherein the applicator and contents thereof are sterilized.

18. The tissue adhesive composition kit according to any of claim 15, wherein the applicator is sterilized to provide a sterility assurance level (SAL) of at least 10⁻³ or 10⁻⁶, 10⁻¹² as measured according to an appropriate validation method.

19. The tissue adhesive composition kit according to claim 16, wherein the silicone elastomer comprises polydimethylsiloxane, and wherein the alkyl cyanoacrylate is one or more of butyl cyanoacrylate and 2-octyl cyanoacrylate.

20. A method for preparing a kit suitable for applying a tissue adhesive, comprising the steps of:
obtaining the tissue adhesive composition according to claim 1;
adding the tissue adhesive composition to a container;
sealing the container;
placing the sealed container in an applicator;
sterilizing the applicator and contents thereof;
sealing the sterilized applicator within a kit container.
